(19) United States
(12) Patent Application Publication
(10) Pub. No.: US 2010/0173983 A1
(43) Pub. Date: Jul. 8, 2010

(54) METHOD FOR INDUCING AUTOPHAGY

(76) Inventors: David Brown, New South Wales (AU); Alan James Husband, New South Wales (AU); Gil Mor, New Haven, CT (US)

Correspondence Address: Pearl Cohen Zedek Latzer, LLP, 1500 Broadway, 12th Floor, New York, NY, 10036, US

(21) Appl. No.: 12/531,339

(22) PCT Filed: Mar. 5, 2008
(86) PCT No.: PCT/AU08/00286

§ 371 Date: Dec. 16, 2009

Related U.S. Application Data
(60) Provisional application No. 60/918,365, filed on Mar. 16, 2007.

(51) Int. Cl. A61K 31/353; A61P 21/00; A61P 25/00; A61P 9/10

(52) U.S. Cl. 514/456

(57) ABSTRACT

The present invention relates to methods for inducing or promoting autophagy in a cell, the method comprising exposing to the cell an effective amount of a compound of formula I as described herein. The invention also relates to methods for treating or preventing diseases and disorders by administering to subjects in need thereof an effective amount of a compound of formula I, wherein the compound induces or promotes autophagy in at least one cell of the subject.

### FIELD OF THE INVENTION

The present invention relates generally to methods for inducing or promoting autophagy and to the treatment of diseases and conditions associated with defective autophagy or autophagic processes.

### BACKGROUND OF THE INVENTION

Autophagy is a highly regulated intracellular pathway for the degradation and turnover of cellular constituents, in particular organelles and proteins. Autophagy plays an important physiological role in the maintenance of cellular homeostasis, as an adaptation (or cryoprotective response) during periods of nutrient deprivation or other stress. Autophagy enables the recycling of amino acids and prevents oxidative stress by promoting the removal of damaged organelles, thereby allowing cellular remodelling. Autophagy also plays essential roles in development, differentiation and tissue remodelling. Three predominant forms of autophagy have been described in mammalian cells—microautophagy, macroautophagy and chaperone-mediated autophagy. Of these, macroautophagy, the bulk lysosomal degradation of large cytoplasmic proteins and organelles, is the major catabolic pathway for the degradation and turnover of macromolecules in mammals.

One of the essential autophagy genes is Beclin 1. (BECN1). BECN1, a mammalian homologue of yeast Atg6/Vps30 necessary to induce autophagy in response to nitrogen deprivation, was identified as a BCL2-interacting gene product. Monoallelic loss of this gene (Becn1−/+) increases the incidence of lung cancer, hepatocellular carcinoma, and lymphoma in experimental animals indicating that that inhibition of autophagy (via targeting BECN1) could provide tumours with some developmental advantages.

The molecular regulation of autophagy occurs on many levels. For example, growth factor bound receptor signalling causes the activation of class I phosphatidylinositol 3-phosphate kinase (PI3K) at the plasma membrane thereby activating its downstream targets AKT and mTOR, and preventing the induction of autophagy. Overexpression of the phosphatase and tensin homologue (PTEN) gene, by an inducible promoter, antagonizes class I PI3K to induce autophagy. Complexation of class III PI3K with BECN1 at the trans-Golgi network also causes the induction of autophagy. Further, downregulation of BCL2, or upregulation of BCL2-adenovirus E1B 19-kD-interacting protein 3 (BNIP3) or HSPIN1 at the mitochondria, also induces autophagy. Additionally, autophagy can also be induced by the cell death-associated protein kinase (DAPK) and the death associated related protein kinase 1 (DRP1).

In addition to its homeostatic function, autophagy has increasingly been implicated in a variety of disorders and disease conditions. For example, defective autophagy is now recognised as a causative factor in pathological conditions such as muscular disorders (vacuolar myopathies), neurodegenerative diseases, liver disease, infections by pathogens and some cancers (see for example Kelekar, 2005 and Nixon, 2006). In the case of chronic neurodegenerative diseases, it is now recognised that autophagy plays a cryoprotective role and that inadequate or defective autophagy promotes neuronal cell death. In some instances the efficiency of autophagy decreases with age, contributing further to neural cell death in diseases such as Alzheimer's Disease and Parkinson's Disease (Nixon, 2006). Further, autophagy can also function as a protective mechanism against infection by bacteria and viruses. Recent findings also suggest that autophagy may occur in advanced atherosclerotic plaques and is thought to be initiated in plaque smooth muscle cells as a result of cellular distress (Schrijvers et al, 2007). In view of the role of smooth muscle cells in promoting plaque stability, autophagic smooth muscle cells in the fibrous cap of atherosclerotic plaque may reflect an important feature underlying plaque stability. Further, in a swine model of chronic myocardial ischemia it has been demonstrated that the cells of an ischemic myocardium are hypervacuolated and that ischemic myocardium tissue has increased expression of proteins knows to be involved in autophagy e.g., beclin 1, cathepsins B and D, heat shock cognate protein (Hsc73), and the processed form of microtubule-associated protein 1 light chain 3 (LC3)). The conclusion of this study was that autophagy triggered by ischemia could be a homeostatic mechanism used by myocardial cells to prevent apoptosis and limit the effects of chronic ischemia.

Accordingly, there is increasing interest in the therapeutic application of autophagy inducers as the modulation of autophagy shows promise as a potential therapeutic approach for the treatment of a variety of human diseases.

As herein described, the present inventors have identified a class of isoflavonoid compounds which induce autophagy in human cells, thereby opening up a range of potential therapeutic avenues.

### SUMMARY OF THE INVENTION

According to a first aspect of the present invention, there is provided a method for inducing or promoting autophagy in a cell, the method comprising exposing to the cell an effective amount of a compound of formula (I)

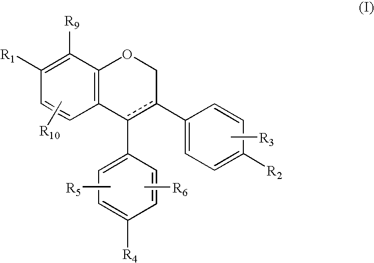

wherein
R₁ is hydrogen, hydroxy, alkyl, alkoxy, halo or OC(O)R₇,
R₂ and R₃ are independently hydrogen, hydroxy, alkoxy, alkyl, cycloalkyl, halo or OC(O)R₇,
R₄, R₅ and R₆ are independently hydrogen, hydroxy, alkoxy, alkyl, cycloalkyl, acyl, amino, C₁₋₄-alkylamino or di(C₁₋₄alkyl)amino, OC(O)R₇ or OR₈,
R₇ is hydrogen, alkyl, cycloalkyl, aryl, arylalkyl or amino, and
R₈ is aryl or arylalkyl,
R₉ and R₁₀ are independently hydrogen, hydroxy, alkyl, alkoxy or halo, and the drawing [symbol] represents a single bond or a double bond,
   or a pharmaceutically acceptable salt or derivative thereof.

In one embodiment, the compound is 3-(4-hydroxyphenyl)-4-(4-methoxyphenyl)-8-methylchroman-7-ol.

Exposure of the cell to the compound may occur in vitro, ex vivo or in vivo.

In a particular embodiment the cell is not a cancer cell. In an embodiment the cell may be selected from a neuronal cell, myocardial cell, muscle cell or liver cell.

According to a second aspect of the present invention, there is provided a method for the treatment or prevention of a disease or disorder, the method comprising administering to a subject in need thereof an effective amount of a compound of formula (I) as described herein, or a pharmaceutically acceptable salt or derivative thereof, optionally in association with one or more pharmaceutically acceptable diluents, adjuvants and/or excipients, wherein the compound induces or promotes autophagy in at least one cell of the subject.

In a particular embodiment the cell is not a cancer cell. In an embodiment the cell may be selected from a neuronal cell, myocardial cell, muscle cell or liver cell.

Typically the disease or disorder is associated with defective, impaired or otherwise aberrant autophagy or autophagic processes.

In an embodiment, the disease or disorder may be selected from a neurodegenerative disease, atherosclerosis, ischemia, a liver disease, a muscle disorder (such as a vacuolar myopathy) or a viral or bacterial infection.

The cell may be a neuronal cell and the method may comprise preventing neuronal cell death.

The cell may be a smooth muscle cell and the method may comprise maintaining atherosclerotic plaque stability.

In one embodiment, the compound is 3-(4-hydroxyphenyl)-4-(4-methoxyphenyl)-8-methylchroman-7-ol.

According to a third aspect of the present invention, there is provided an agent for the treatment or prevention of a disease or disorder, the agent comprising a compound of formula (I) as described herein, or a pharmaceutically acceptable salt or derivative thereof.

According to a fourth aspect of the present invention, there is provided the use of a compound of formula (I) as described herein for the manufacture of a medicament for inducing or promoting autophagy in a cell.

According to a fifth aspect of the present invention, there is provided the use of a compound of formula (I) as described herein for the manufacture of a medicament for treating or preventing a disease or disorder, wherein the compound induces or promotes autophagy in at least one cell of the subject.

Typically in accordance with the above aspects and embodiments the subject is human. In other embodiments, the subject may be selected from the group consisting of, but not limited to: primate, ovine, bovine, canine, feline, porcine, equine and murine.

### BRIEF DESCRIPTION OF THE DRAWINGS

The present invention will now be described, by way of non-limiting example only, with reference to the accompanying drawings.

FIG. 1. Comparative cytotoxicity of dehydroequol and Compound I as described herein (Cpd 1) against normal and ovarian cancer cells: CP70 (cell line), R179, R182, R585 (primary ovarian cancer explants) and OSE (normal ovarian surface epithelial cells). Cpd 1 decreased cell viability of all primary ovarian cancer cells and also exhibits some toxicity against normal OSE cells. Dehydroequol does not exhibit this pan activity. Both compounds were tested at concentrations up to 10 μg/ml and exposed to cells for 24 hr.

FIG. 2. Comparative induction of caspase 3 in R182 ovarian cancer explants by Cpd 1 and dehydroequol using either dose response (A) or duration of exposure (B). Caspase 3 activity was assessed using the Caspase-Glo 3/7 assays. Dehydroequol-induced activation of caspase 3 is time (5 μg/ml) and dose dependent (0-25 hr). Cpd 1 did not induce caspase 3 activity.

FIG. 3. Comparative induction of caspase 9 in R182 ovarian cancer explants by Cpd 1 and dehydroequol using either dose response (A) or duration of exposure (B). Caspase 9 activity was assessed using the Caspase-Glo 9 assay. Dehydroequol-induced activation of caspase 3 is time (5 μg/ml) and dose dependent (0-25 hr). Cpd 1 did not induce caspase 9 activity. Similar results were observed with other EOC cells tested.

FIG. 4. Comparative induction of caspase 8 in R182 ovarian cancer explants by Cpd 1 and dehydroequol using either dose response (A) or duration of exposure (B). Caspase 8 activity was assessed using the Caspase-Glo 8 assay. Dehydroequol-induced activation of caspase 8 is time (5 μg/ml) and dose dependent (0-25 hr). Cpd 1 did not induce caspase 8 activity.

FIG. 5. Comparative cell viability plots of dehydroequol (A) and Cpd 1 (B) treated cells in the presence and absence of the pan caspase inhibitor ZVAD-FMK (0.5 μg/ml). Where Cpd 1-induced cell death proceeded in the presence of ZVAD-FMK, dehydroequol-induced apoptosis was inhibited demonstrating the Cpd 1-induced cell death is proceeding via a caspase independent pathway.

FIG. 6. Phase-contrast images of R182 cells treated with vehicle (NT) or 5 μg/ml Cpd 1 over 4 hr and 8 hr (A). (B), shows R-182 cells treated with Cpd 1 (5 μg/ml) after 8 hr at higher magnification (×50). Vacuole formation is demonstrated by the red arrows.

FIG. 7. Cpd 1 induces DNA degradation in CP70 cells. A, No treatment control. B, Cpd 1-treated cells. CP70 ovarian cancer cells were incubated with 10 μg/ml Cpd 1 for 12 hr and fixed. Cells were co-stained with propidium iodide and Hoechst A and incidence of PI labelled cells indicative of DNA degradation in quandrant 2 was assessed by FACS analysis.

FIG. 8. Molecular evidence of autophagic cell death in R-182 ovarian cancer cells induced by Cpd 1. A, Western blot analysis on lysate preparations (20 μg protein) of R-182 cells treated with Cpd 1 (5 μg/ml) over 24 hr demonstrated that LC3-II expression is upregulated in cytoplasmic preparations. XIAP expression remains unchanged. Mitochondrial preparations show that Beclin 1 is transiently upregulated after 1 hr exposure and Bax is stably upregulated 4-24 hr post treatment. Nuclear preparations from the same cell population also show that endonuclease G is also upregulated. B, Beclin 1 binds to Bcl-2. Immunoprecipitation study using mitochondrial preparations of cells pre-treated with Cpd 1 as stated in A, and subjected to beclin 1 immunoprecitation. Immunoprecipitated fractions were washed in PBS, subjected to SDS-PAGE, blotted and then probed with a mouse anti-Bcl-2 antibody. Actin and Topo-1 were used as loading control. C, Depolarized cells resulting from over-expression of Bax and Beclin 1 following administration of Cpd 1. Cells were treated with Cpd 1 as described in A, pelleted and washed in HBSS and the cell pellet resuspended in the reaction buffer for JC-1 (Biovision, Inc. MitoCapture™ K250-100). HBSS-washed cells were analysed by flow cytometry (λ_{excit}=488 nm and (λₑₘᵢₜ=530 nm for green fluorescence (depolarized) or 590 nm for red fluorescence (polarized). 10,000 gated events were measured with a BD FACScalibur. Results were expressed as mean±standard deviation (n=3).

FIG. 9. Cpd 1-induced cell death involves cytochrome c translocation to the cytoplasm. R-182 cells treated with Cpd 1 (5 μg/ml) over 8 hr and the cytoplasmic and mitochondrial fractions were separated. Western blot analyses of cytoplasmic extracts demonstrate that the occurrence of cytochrome in the cytoplasm is increased over time when compared to no treatment controls. Cox-4 was included to show the integrity of the mitochondrial preparation. Similar results were observed with other EOC cells tested.

FIG. 10. The expression of upstream regulators of autophagy are also modulated in response to Cpd 1. A, Western blot analysis on lysate preparations (20 μg protein) of R-182 cells treated with Cpd 1 (5 μg/ml) over 24 hr demonstrated that both mTOR expression and Akt phosphorylation status (p-Akt) is reduced up to 120 minutes post exposure to Cpd 1. Total Akt (t-Akt) expression remains unchanged. B-actin was included as a loading control. B, Proposed mechanism of Cpd 1 induced autophagic cell death.

FIG. 11. Cpd 1 markers of autophagy observed in in vitro studies are also present in vivo. Immunohistological analysis of Paraffin sections (5 μm) of ovarian cancer xenograft tissue confirm that endonuclease G was upregulated in tumours excised from Cpd 1 (100 mg/kg) treated mice (A) compared to control (B). C, The phosphorylation status of S6K, a downstream mediator of mTOR, was downregulated in tumour samples. T-S6K served as loading control.

### DETAILED DESCRIPTION OF THE INVENTION

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

The articles “a” and “an” are used herein to refer to one or to more than one (i.e., to at least one) of the grammatical object of the article. By way of example, “an element” means one element or more than one element.

As used herein the terms “treating”, “treatment”, “preventing” and “prevention” refer to any and all uses which remedy a condition or symptoms, prevent the establishment of a condition or disease, or otherwise prevent, hinder, retard, or reverse the progression of a condition or disease or other undesirable symptoms in any way whatsoever. Thus the terms “treating” and “preventing” and the like are to be considered in their broadest context. For example, treatment does not necessarily imply that a patient is treated until total recovery.

As used herein the terms “effective amount” and “effective dose” include within their meaning a non-toxic but sufficient amount or dose of an agent or compound to provide the desired effect. The exact amount or dose required will vary from subject to subject depending on factors such as the species being treated, the age and general condition of the subject, the severity of the condition being treated, the particular agent being administered and the mode of administration and so forth. Thus, it is not possible to specify an exact “effective amount” or “effective dose”. However, for any given case, an appropriate “effective amount” or “effective dose” may be determined by one of ordinary skill in the art using only routine experimentation.

The term “pharmaceutically acceptable salt” refers to an organic or inorganic moiety that carries a charge and that can be administered in association with a pharmaceutical agent, for example, as a counter-cation or counter-anion in a salt. Pharmaceutically acceptable cations are known to those of skilled in the art, and include but are not limited to sodium, potassium, calcium, zinc and quaternary amine. Pharmaceutically acceptable anions are known to those of skill in the art, and include but are not limited to chloride, acetate, citrate, bicarbonate and carbonate.

The term “pharmaceutically acceptable derivative” refers to a derivative of the active compound that upon administration to the recipient, is capable of providing directly or indirectly, the parent compound or metabolite, or that exhibits activity itself. Prodrugs are included within the scope of the present invention.

Programmed cell death typically proceeds via caspase-mediated apoptosis. Morphologically, cells undergoing apoptosis display blebbing, condensed chromatin, and contain apoptotic bodies. In contrast, autophagy is a caspase-independent process in which intracellular vacuoles termed autolysosomes sequester and degrade proteins, and organelles thereby enabling the recycling of macromolecules. As such autophagy plays an important physiological role in the maintenance of cellular homeostasis, tissue re-modelling, cellular differentiation and development, and as an adaptive process in response to stress. The autophagic cell death pathway is typically invoked in response to stress or other signals either independently of apoptosis or when the apoptotic cascade is non-functional thereby providing an alternative druggable pathway that can be manipulated to circumvent chemoresistance. Molecular markers of autophagy include, beclin 1, cathepsins B and D, heat shock cognate protein (Hsc73), and the processed form of microtubule-associated protein 1 light chain 3 (LC3) (Kondo et al., 2005).

As exemplified herein the present inventors have surprisingly found that an isoflavonoid compound, herein designated compound 1 (Cpd 1) induces cell death via the autophagic pathway in human cells. Cell death is apoptosis-independent, involving endonuclease G translocation to the nucleus resulting in DNA degradation and vacuolated cells. This compound does not upregulate the activity of caspases 3, 8 and 9 whilst markers of autophagy, including beclin-1 and LC3-II, are upregulated. That Cpd 1 induces autophagic cell death has also been confirmed by in vivo studies.

In one aspect, the present invention provides a method for inducing or promoting autophagy in a cell, the method comprising exposing to the cell and effective amount of a compound of formula I. The present invention also provides methods for the treatment or prevention of diseases and disorders associated with reduced or otherwise aberrant autophagy. Accordingly, one aspect of the invention provides a method for preventing or treating a disease or disorder in a subject, the method comprising administering to the subject an effective amount of a compound of formula I, wherein the compound induces or promotes autophagy in at least one cell of the subject. Optionally, the compound is administered in the form of a pharmaceutical composition, which composing may comprise one or more pharmaceutically acceptable diluents, adjuvants and/or excipients.

It will also be readily appreciated by those skilled in the art that the present invention contemplates the administration of more than one compound of formula I, and/or the administration of at least one compound of formula I in conjunction with at least one additional therapeutic compound or agent.

Compounds useful in the present invention are of the general formula (I):

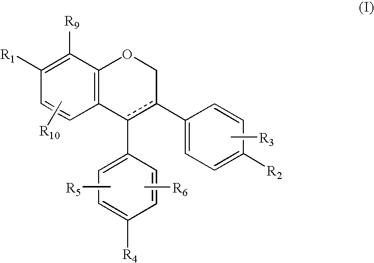

wherein
R₁ is hydrogen, hydroxy, alkyl, alkoxy, halo or OC(O)R₇,
R₂ and R₃ are independently hydrogen, hydroxy, alkoxy, alkyl, cycloalkyl, halo or OC(O)R₇,
R₄, R₅ and R₆ are independently hydrogen, hydroxy, alkoxy, alkyl, cycloalkyl, acyl, amino, C₁₋₄-alkylamino or di(C₁₋₄-alkyl)amino, OC(O)R₇ or OR₈,
R₇ is hydrogen, alkyl, cycloalkyl, aryl, arylalkyl or amino, and
R₈ is aryl or arylalkyl,
R₉ and R₁₀ are independently hydrogen, hydroxy, alkyl, alkoxy or halo, and the drawing [symbol] represents a single bond or a double bond,
   or a pharmaceutically acceptable salt or derivative thereof.

Preferably in compounds of formula (I) the substitution pattern of R₂ and R₃ is as shown below:

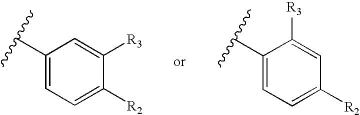

Preferably in compounds of formula (I) the substitution pattern of R₄, R₅ and R₆ is as shown below:

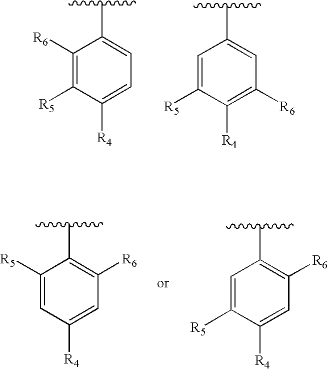

Preferably in compounds of formula (I) the drawing [symbol] represents a single bond.

Preferably in compounds of formula (I):
R₁ is hydroxy, C₁₋₄-alkoxy or OC(O)R₇,
R₂ and R₃ are independently hydrogen, hydroxy, C₁₋₄-alkoxy, halo or OC(O)R₇,
R₄, R₅ and R₆ are independently hydrogen, hydroxy, alkoxy, alkyl, cycloalkyl, acyl, OC(O)R₇, and
R₇ is C₁₋₄-alkyl, phenyl or benzyl,
R₉ is hydrogen, hydroxy, alkyl or halo,
   or a pharmaceutically acceptable salt or derivative thereof.

More preferably in compounds of formula (I):
R₁ is hydroxy, methoxy, ethoxy or acetyloxy,
R₂ and R₃ are independently hydrogen, hydroxy, methoxy, ethoxy, propoxy, isopropoxy, bromo, chloro, fluoro or acetyloxy,
R₄ is hydrogen, hydroxy, methoxy, ethoxy, propoxy, isopropoxy or acetyloxy, and
R₅ and R₆ are independently hydrogen, hydroxy, methoxy, ethoxy, propoxy, isopropoxy, acetyl, or acetyloxy,
R₉ is hydrogen, hydroxy, methyl, methoxy, bromo, chloro, fluoro or acetyloxy,
R₁₀ is hydrogen,
   or a pharmaceutically acceptable salt or derivative thereof.

Preferred compounds of formula (I) have the following substituents where:
R₁ is hydroxy, methoxy or acetyloxy,
R₂ and R₃ are independently hydrogen, hydroxy, methoxy, bromo or acetyloxy,
R₄ and R₆ are independently hydrogen, hydroxy, methoxy or acetyloxy,
R₅ and R₁₀ are hydrogen, and
R₉ is hydrogen, methyl or bromo,
   or a pharmaceutically acceptable salt or derivative thereof.

In a preferred embodiment, R₉ is methyl.

Preferred compounds of formula (I) include:
3-(4-hydroxyphenyl)-4-(4-methoxyphenyl)-8-methylchroman-7-ol (Cpd. 1);
3-(4-methoxyphenyl)-4-(4-methoxyphenyl)-7-methoxy-8-methylchroman (Cpd. 2);
3-(3,4-dimethoxyphenyl)-4-(4-methoxyphenyl)-8-methylchroman-7-ol (Cpd. 3);
3-(4-methoxyphenyl)-4-(4-methoxyphenyl)-8-methylchroman-7-ol (Cpd. 4);
3-(4-hydroxyphenyl)-4-(4-methoxyphenyl)-7-methoxy-8-methylchroman (Cpd. 5);
3-(3-methoxyphenyl)-4-(4-methoxyphenyl)-8-methylchroman-7-ol (Cpd. 6);
3-(3,4-dihydroxyphenyl)-4-(4-methoxyphenyl)-7-methoxy-8-methylchroman (Cpd. 7);
3-(3-hydroxyphenyl)-4-(4-methoxyphenyl)-8-methylchroman-7-ol (Cpd. 8);
3-(3,4-dihydroxyphenyl)-4-(4-methoxyphenyl)-8-methylchroman-7-ol (Cpd. 9);
   or a pharmaceutically acceptable salt thereof.

In another preferred embodiment, R₉ is hydrogen.

Preferred compounds of formula (I) include:
3-(4-hydroxyphenyl)-4-(4-methoxyphenyl)chroman-7-ol (Cpd. 10);
3-(4-hydroxyphenyl)-4-phenylchroman-7-ol (Cpd. 11);
3-(4-hydroxyphenyl)-4-(3-methoxyphenyl)chroman-7-ol (Cpd. 12);
3-(3,4-dimethoxyphenyl)-4-(4-methoxyphenyl)chroman-7-ol (Cpd. 13);
3-(4-hydroxyphenyl)-4-(4-methylphenyl)chroman-7-ol (Cpd. 14);
3-(4-methoxyphenyl)-4-(4-methoxyphenyl)-7-methoxychroman (Cpd. 15);
3-(4-hydroxyphenyl)-4-(2,6-dimethoxy-4-hydroxyphenyl)chroman-7-ol (Cpd. 16);
3-(4-hydroxyphenyl)-4-(2-hydroxyphenyl)chroman-7-ol (Cpd. 17);
3-(4-hydroxyphenyl)-4-(3-acyl-2-hydroxy-4-methoxyphenyl)chroman-7-ol (Cpd. 18);
3-(3-hydroxyphenyl)-4-(3-methoxyphenyl)chroman-7-ol (Cpd. 19);
3-(4-hydroxyphenyl)-4-(4-hydroxyphenyl)chroman-7-ol (Cpd. 20);
3-(4-bromophenyl)-4-(4-methoxyphenyl)chroman-7-ol (Cpd. 21);
3-(4-hydroxyphenyl)-4-(3-methoxyphenyl)chroman-7-ol (Cpd. 22);
3-(4-hydroxyphenyl)-4-(3-aminophenyl)chroman-7-ol (Cpd. 23);
3-(4-hydroxyphenyl)-4-(4-phenoxyphenyl)chroman-7-ol (Cpd 24);
   or a pharmaceutically acceptable salt thereof.

The compounds of formula (I) according to the invention include two chiral centres. The present invention includes all the enantiomers and diastereoisomers as well as mixtures thereof in any proportions. The invention also extends to isolated enantiomers or pairs of enantiomers. Methods of separating enantiomers and diastereoisomers are well known to person skilled in the art.

It will be clear to persons skilled in the art that the in compounds of formula (I) the aryl substituents on the heterocyclic ring can be cis or trans relative to each other. Preferably in the compounds of formula (I) these substituents will be cis.

A preferred compound of the present invention is the cis-isomer of compound No 1 (Cpd 1):

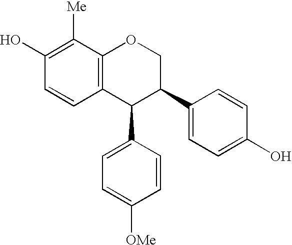

or a pharmaceutically acceptable salt thereof.

Similarly, preferred compounds are compound Nos. (2) to (24) in the cis-conformation.

The term “alkyl” is taken to include straight chain and branched chain saturated alkyl groups of 1 to 6 carbon atoms, such as methyl, ethyl, propyl, isopropyl, butyl, isobutyl, secbutyl, tertiary butyl, pentyl and the like. The alkyl group more preferably contains preferably from 1 to 4 carbon atoms, especially methyl, ethyl, propyl or isopropyl.

Cycloalkyl includes C₃₋₆ cycloalkyl such as cyclopropyl, cyclobutyl, cyclopentyl and cyclohexyl.

The alkyl group or cycloalkyl group may optionally be substituted by one or more of fluorine, chlorine, bromine, iodine, carboxyl, C₁-C₄-alkoxycarbonyl, C₁-C₄-alkylamino-carbonyl, carbonyl, hydroxyl, C₁-C₄-alkoxy, formyloxy, C₁-C₄-alkyl-carbonyloxy, C₃-C₆ cycloalkyl or phenyl.

Preferably the alkyl group does not bear any substituents.

The term “aryl” is taken to include phenyl, benzyl, biphenyl and naphthyl and may be optionally substituted by one or more C₁-C₄-alkyl, hydroxy, C₁-C₄-alkoxy, carbonyl, C₁-C₄-alkoxycarbonyl, C₁-C₄-alkylcarbonyloxy, nitro or halo.

The term “halo” is taken to include fluoro, chloro, bromo and iodo, preferably fluoro and chloro, more preferably fluoro. Reference to for example “haloalkyl” will include monohalogenated, dihalogenated and up to perhalogenated alkyl groups. Preferred haloalkyl groups are trifluoromethyl and pentafluoroethyl.

The compounds of the invention include all salts, such as acid addition salts, anionic salts and zwitterionic salts, and in particular include pharmaceutically acceptable salts as would be known to those skilled in the art. Pharmaceutically acceptable salts include those formed from: acetic, ascorbic, aspartic, benzoic, benzenesulphonic, citric, cinnamic, ethanesulphonic, fumaric, glutamic, glutaric, gluconic, hydrochloric, hydrobromic, lactic, maleic, malic, methanesulphonic, naphthoic, hydroxynaphthoic, naphthalenesulphonic, naphthalenedisulphonic, naphthaleneacrylic, oleic, oxalic, oxaloacetic, phosphoric, pyruvic, p-toluenesulphonic, tartaric, trifluoroacetic, triphenylacetic, tricarballylic, salicylic, sulphuric, sulphamic, sulphanilic and succinic acid.

Pharmaceutically acceptable derivatives include solvates, pharmaceutically active esters, prodrugs or the like. This also includes derivatives with physiologically cleavable leaving groups that can be cleaved in vivo to provide the compounds of the invention or their active moiety. The leaving groups may include acyl, phosphate, sulfate, sulfonate, and preferably are mono-, di- and per-acyl oxy-substituted compounds, where one or more of the pendant hydroxy groups are protected by an acyl group, preferably an acetyl group. Typically acyloxy substituted compounds of the invention are readily cleavable to the corresponding hydroxy substituted compounds.

Compounds of formula I to which the present invention relates are believed to have favourable toxicity profiles with normal cells and good bioavailability. These compounds are described in International Patent Applications PCT/AU2005/001435 (published as WO 2006/032085) and PCT/AU2005/001436 (published as WO 2006/032086), the disclosures of which are incorporated herein by reference.

Embodiments of the present invention find particular application in the therapeutic or prophylactic treatment of diseases and disorders which are associated with reduced, impaired or otherwise aberrant autophagy or autophagic processes. By way of examples, diseases and disorders in which methods of the present invention find particular application include, but are not limited to, neurodegenerative disease such as Alzheimer's Disease, Huntington's Disease and Parkinson's Disease, muscular disorders, liver disease, pathogen infection and cardiovascular diseases such as atherosclerosis and myocardial ischemia. In atherosclerosis, compounds of the present invention find application, for example, in the induction or promotion of autophagy in smooth muscle cells in the fibrous cap of atherosclerotic plaques, whereby the induction or promotion of autophagy assists in maintaining plaque stability.

According to the methods of present invention isoflavonoid compounds and compositions comprising such isoflavonoids may be administered by any suitable route, either systemically, regionally or locally. The particular route of administration to be used in any given circumstance will depend on a number of factors, including the nature of the condition to be treated, the severity and extent of the condition, the required dosage of the particular compound to be delivered and the potential side-effects of the compound. For example, in circumstances where it is required that appropriate concentrations of the desired compound are delivered directly to the site in the body to be treated, administration may be regional rather than systemic. Regional administration provides the capability of delivering very high local concentrations of the desired compound to the required site and thus is suitable for achieving the desired therapeutic or preventative effect whilst avoiding exposure of other organs of the body to the compound and thereby potentially reducing side effects.

By way of example, administration according to embodiments of the invention may be achieved by any standard routes, including intracavitary, intravesical, intramuscular, intraarterial, intravenous, intraocular, subcutaneous, topical or oral.

In employing methods of the invention, isoflavonoid compounds may be formulated in pharmaceutical compositions. Suitable compositions may be prepared according to methods which are known to those of ordinary skill in the art and may include a pharmaceutically acceptable diluent, adjuvant and/or excipient. The diluents, adjuvants and excipients must be “acceptable” in terms of being compatible with the other ingredients of the composition, and not deleterious to the recipient thereof. The diluent, adjuvant or excipient may be a solid or a liquid, or both, and may be formulated with the compound as a unit-dose, for example, a tablet, which may contain from 0.5% to 59% by weight of the active compound, or up to 100% by weight of the active compound. One or more active compounds may be incorporated in the formulations of the invention, which may be prepared by any of the well known techniques of pharmacy consisting essentially of admixing the components, optionally including one or more accessory ingredients.

Examples of pharmaceutically acceptable diluents are demineralised or distilled water; saline solution; vegetable based oils such as peanut oil, safflower oil, olive oil, cottonseed oil, maize oil, sesame oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 1% to 99.9% by weight of the compositions.

Formulations suitable for oral administration may be presented in discrete units, such as capsules, sachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture such as to form a unit dosage. For example, a tablet may be prepared by compressing or moulding a powder or granules containing the active compound, optionally with one or more accessory ingredients. Compressed tablets may be prepared by compressing, in a suitable machine, the compound of the free-flowing, such as a powder or granules optionally mixed with a binder, lubricant, inert diluent, and/or surface active/dispersing agent(s). Moulded tablets may be made by moulding, in a suitable machine, the powdered compound moistened with an inert liquid binder.

Solid forms for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Liquid forms for oral administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof.

Formulations suitable for buccal (sublingual) administration include lozenges comprising the active compound in a flavoured base, usually sucrose and acacia or tragacanth; and pastilles comprising the compound in an inert base such as gelatin and glycerin or sucrose and acacia.

Compositions of the present invention suitable for parenteral administration typically conveniently comprise sterile aqueous preparations of the active compounds, which preparations may be isotonic with the blood of the intended recipient. These preparations are typically administered intravenously, although administration may also be effected by means of subcutaneous, intramuscular, or intradermal injection. Such preparations may conveniently be prepared by admixing the compound with water or a glycine buffer and rendering the resulting solution sterile and isotonic with the blood. Injectable formulations according to the invention generally contain from 0.1% to 60% w/v of active compound(s) and are administered at a rate of 0.1 ml/minute/kg or as appropriate.

Formulations for infusion, for example, may be prepared employing saline as the carrier and a solubilising agent such as a cyclodextrin or derivative thereof. Suitable cyclodextrins include α-cyclodextrin, β-cyclodextrin, γ-cyclodextrin, dimethyl-β-cyclodextrin, 2-hydroxyethyl-β-cyclodextrin, 2-hydroxypropyl-cyclodextrin, 3-hydroxypropyl-β-cyclodextrin and tri-methyl-β-cyclodextrin. More preferably the cyclodextrin is hydroxypropyl-β-cyclodextrin. Suitable derivatives of cyclodextrins include Captisol® a sulfobutyl ether derivative of cyclodextrin and analogues thereof as described in U.S. Pat. No. 5,134,127.

Formulations suitable for rectal administration are typically presented as unit dose suppositories. These may be prepared by admixing the active compound with one or more conventional solid carriers, for example, cocoa butter, and then shaping the resulting mixture.

Formulations or compositions suitable for topical administration to the skin may take the form of an ointment, cream, lotion, paste, gel, spray, aerosol, or oil. Carriers which may be used include Vaseline, lanoline, polyethylene glycols, alcohols, and combination of two or more thereof. The active compound is generally present at a concentration of from 0.1% to 0.5% w/w, for example, from 0.5% to 2% w/w. Examples of such compositions include cosmetic skin creams.

Formulations suitable for inhalation may be delivered as a spray composition in the form of a solution, suspension or emulsion. The inhalation spray composition may further comprise a pharmaceutically acceptable propellant such as carbon dioxide or nitrous oxide or a hydrogen containing fluorocarbon such as 1,1,1,2-tetrafluoroethane, 1,1,1,2,3,3,3-heptafluoro-n-propane or mixtures thereof.

Formulations suitable for transdermal administration may be presented as discrete patches adapted to remain in intimate contact with the epidermis of the recipient for a prolonged period of time. Such patches suitably contain the active compound as an optionally buffered aqueous solution of, for example, 0.1 M to 0.2 M concentration with respect to the said active compound. Formulations suitable for transdermal administration may also be delivered by iontophoresis (see, for example, Pharmaceutical Research 3 (6), 318 (1986)) and typically take the form of an optionally buffered aqueous solution of the active compound. For example, suitable formulations may comprise citrate or bis/tris buffer (pH 6) or ethanol/water and contain from 0.1 M to 0.2 M active ingredient.

The active compounds may be provided in the form of food stuffs, such as being added to, admixed into, coated, combined or otherwise added to a food stuff. The term food stuff is used in its widest possible sense and includes liquid formulations such as drinks including dairy products and other foods, such as health bars, desserts, etc. Food formulations containing compounds of the invention can be readily prepared according to standard practices.

According to the present invention, compounds and compositions may be administered either therapeutically or preventively. In a therapeutic application, compounds and compositions are administered to a patient already suffering from a disease or disorder or experiencing symptoms, in an amount sufficient to cure or at least partially arrest the disease or disorder, symptoms and/or any associated complications. The compound or composition should provide a quantity of the active compound sufficient to effectively treat the patient.

The effective dose level of the administered compound for any particular subject will depend upon a variety of factors including: the type of condition being treated and the stage of the condition; the activity of the compound employed; the composition employed; the age, body weight, general health, sex and diet of the patient; the time of administration; the route of administration; the rate of sequestration of compounds; the duration of the treatment; drugs used in combination or coincidental with the treatment, together with other related factors well known in medicine.

One skilled in the art would be able, by routine experimentation, to determine an effective, non-toxic dosage which would be required to treat applicable conditions. These will most often be determined on a case-by-case basis. By way of example only, an effective dosage may be expected to be in the range of about 0.0001 mg to about 1000 mg per kg body weight per 24 hours; typically, about 0.001 mg to about 750 mg per kg body weight per 24 hours; about 0.01 mg to about 500 mg per kg body weight per 24 hours; about 0.1 mg to about 500 mg per kg body weight per 24 hours; about 0.1 mg to about 250 mg per kg body weight per 24 hours; or about 1.0 mg to about 250 mg per kg body weight per 24 hours. More typically, an effective dose range is expected to be in the range of about 10 mg to about 200 mg per kg body weight per 24 hours.

Further, it will be apparent to those of ordinary skill in the art that the optimal quantity and spacing of individual dosages will principally be determined by the nature and extent of the condition being treated, the form, route and site of administration, and the individual being treated. Suitable conditions can be determined by conventional techniques.

It will also be apparent to those of ordinary skill in the art that the optimal course of treatment, such as, the number of doses of the composition given per day for a defined number of days, can be ascertained by those skilled in the art using conventional course of treatment determination tests.

In accordance with the methods of the invention, isoflavonoid compounds or pharmaceutically acceptable derivatives prodrugs or salts thereof can be co-administered with other active agents that do not impair the desired action, or with agents that supplement the desired action, such as antibiotics, antifungals, antiinflammatories, lipid lowering agents, platelet aggregation inhibitors, antithrombotic agents, calcium channel blockers, corticosteroids or antiviral compounds. The particular agent(s) used will depend on a number of factors and will typically be tailored to the disease or disorder to be treated. The co-administration of agents may be simultaneous or sequential. Simultaneous administration may be effected by the compounds being formulated in a single composition, or in separate compositions administered at the same or similar time. Sequential administration may be in any order as required.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present invention will now be described with reference to the following specific examples, which should not be construed as in any way limiting the scope of the invention.

### EXAMPLES

### Example 1

### Cpd 1 Induces Cell Death Via a Caspase Independent Pathway

Human EOC cell lines A2780, CP70, and OSE were propagated in RPMI plus 10% fetal bovine serum (FBS) (Gemini Bio-Products, Woodland, Calif.) at 37° C. in a 5% CO2 atmosphere. Primary EOC cells (R179, R182, R585) were isolated from malignant ovarian ascites and cultured as previously described (Kamsteeg et al., 2003). Dehydroequol and Compound 1 (Cpd 1) were obtained from Novogen (Australia). All other reagents were purchased from Sigma Chemical (St. Louis, Mo.). The pan caspase inhibitor Z-VAD-FMK was obtained from R&D Systems (Minneapolis, Minn.).

Cell viability was evaluated using the CellTiter 96 Aqueous One Solution Cell Proliferation Assay (Promega, Madison, Wis.) according to the manufacturer's instructions. The values from the treated cells were compared with the values generated from the untreated cells and reported as percent viability. Each experiment was performed in triplicate.

For caspase activity assays, 10 μg of protein in 50 μL total volume was mixed with 50 μL of equilibrated Caspase-Glo 3/7, 8, or 9 reagents (Promega). After incubating at room temperature for 1 hour, luminescence was measured using TD 20/20 Luminometer (Turner Designs, Sunnyvale, Calif.). Blank values were subtracted and fold-increase in activity was calculated based on activity measured from untreated cells. Each sample was measured in triplicate.

Data are expressed as mean±standard deviation (SD). Statistical significance (P<0.05) was determined using both one- and two-way analysis of variance (ANOVA) with Bonferonni correction.

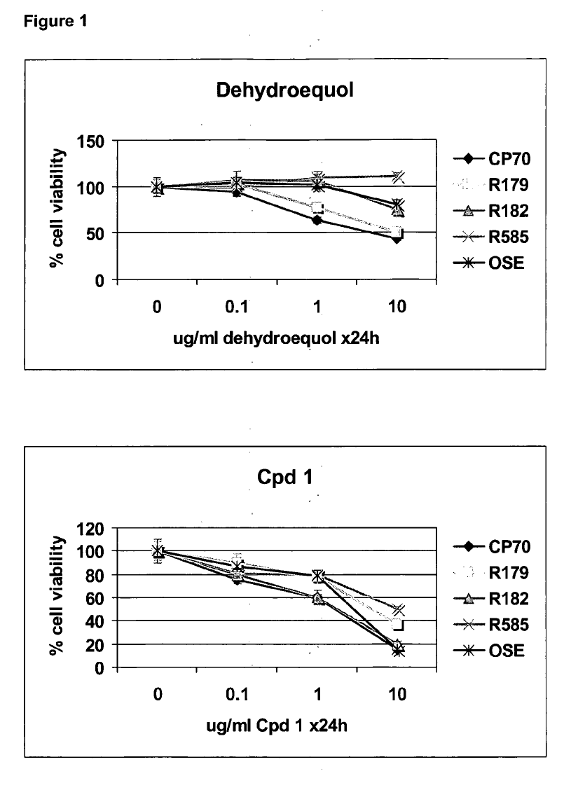

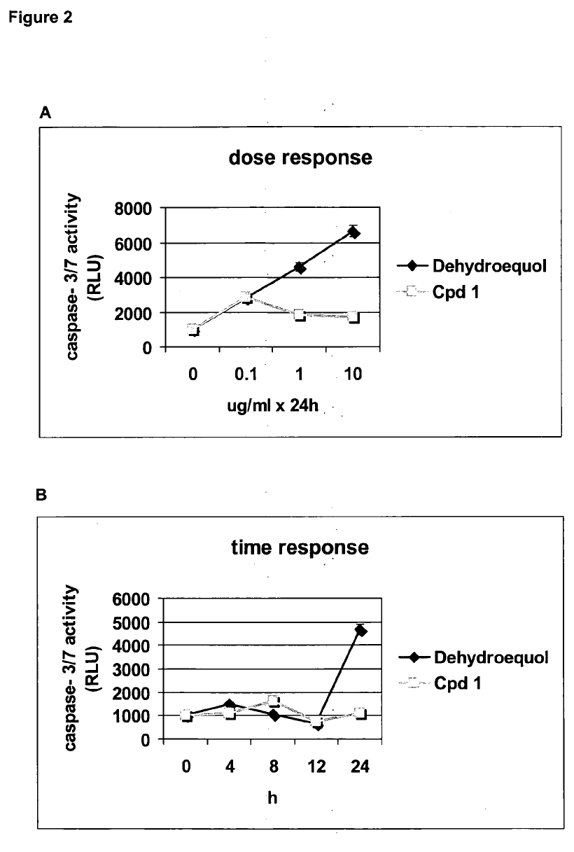

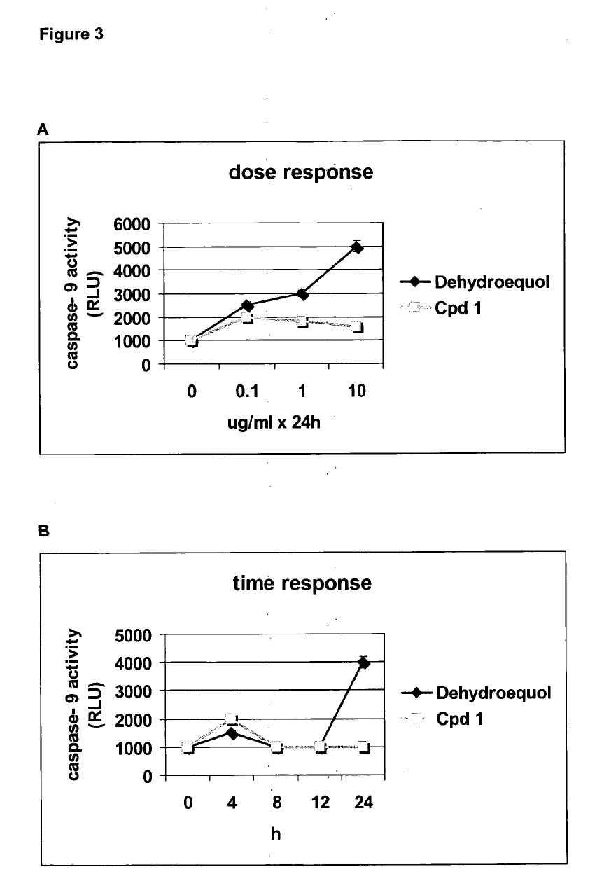

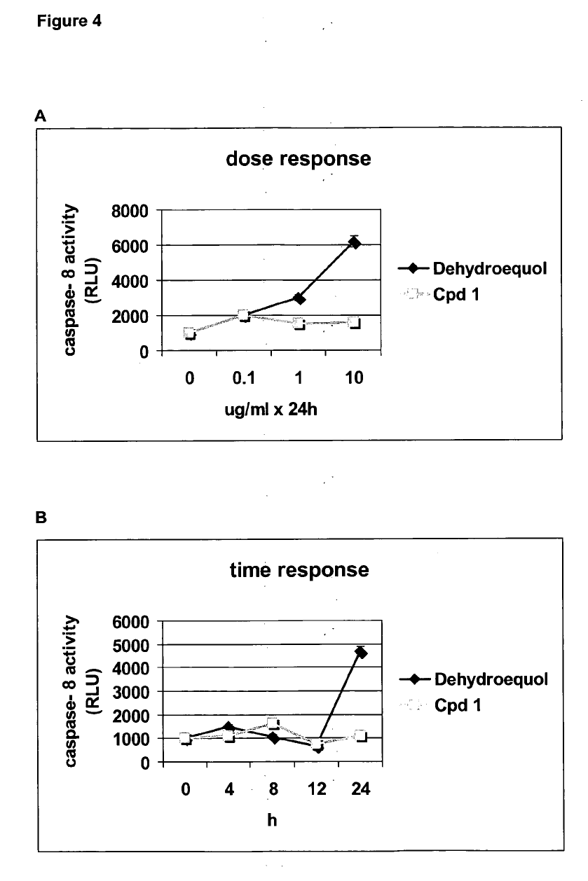

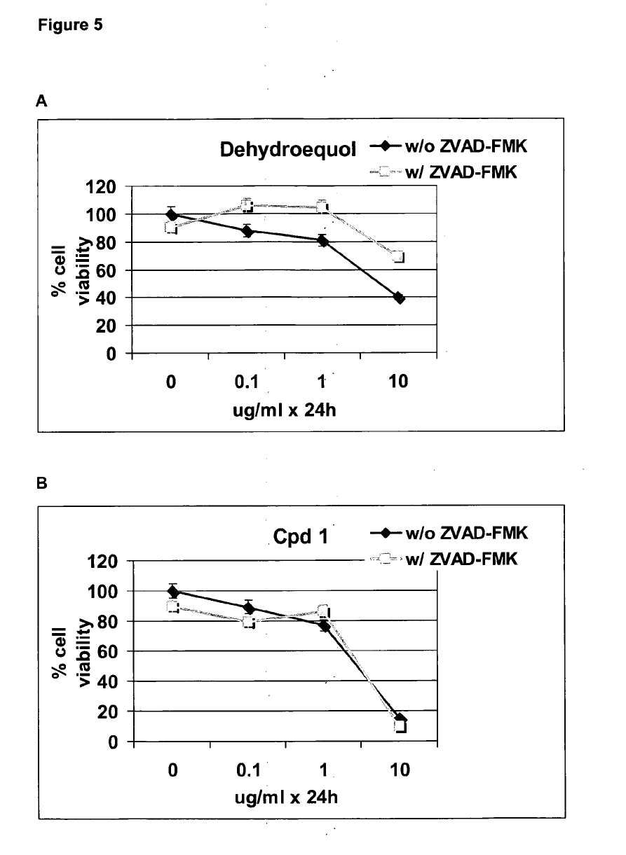

As shown in FIG. 1, Cpd 1 decreased cell viability of all primary ovarian cancer cells and also exhibited some toxicity against normal OSE cells. This cytotoxicity was demonstrated to be caspase independent, a characteristic of autophagy. Cpd 1 administration to R182 cells did not induce activity of caspase 3 (FIG. 2), caspase 9 (FIG. 3) or caspase 8 (FIG. 4). In the presence of the pan caspase inhibitor ZVAD-FMK, Cpd 1-induced cell death proceeded, whereas dehydroequol-induced apoptosis was inhibited (FIG. 5) demonstrating that Cpd 1-induced cell death proceeds via a caspase independent pathway.

### Example 2

### Vacuole Formation and DNA Fragmentation in the Presence of Cpd 1

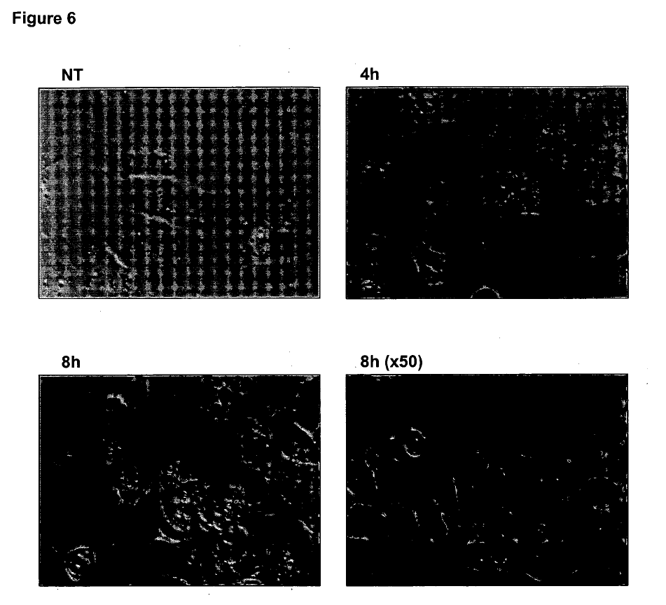

R182 cells treated with Cpd 1 were observed microscopically to determine if morphological and structural alterations to cellular integrity took place. Cells treated with Cpd 1 were hyper-vacuolated (see FIG. 6) and the plasma membrane appeared to bleb or form folds. Phase-contrast images of Cpd 1-treated R182 cells treated with vehicle showed no evidence of vacuole formation, whereas in the presence of 5 μg/ml Cpd 1 over 4 hr and 8 hr vacuole formation is clearly evident (FIG. 6). These morphological changes observed in Cpd 1 treated cells are considered the hall mark of autophagy with the increased vacuoles formation thought to result in autophagosomes which eventually fuse with lysosomes to form autolysosomes. These structures contain catabolic hydrolases which are released into the cytoplasm when the lysosomal membrane becomes compromised.

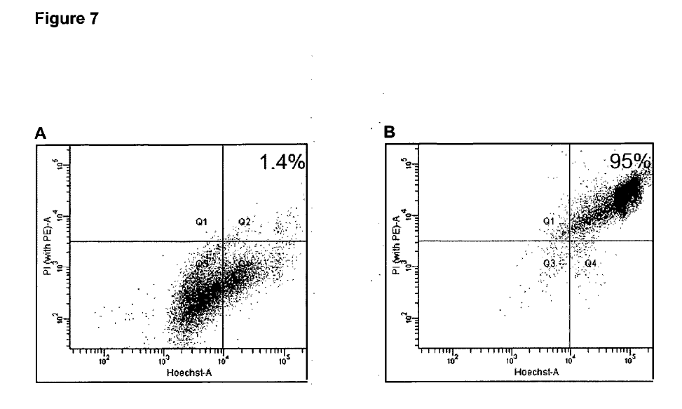

The inventors then determined whether treatment with Cpd 1 induced DNA fragmentation. Ovarian cancer cells were treated with Cpd 1 (10 μM) for 24 hr and gently trypsinised. Trypsinized cells were combined with non-adherent cells, rinsed once in medium containing serum and then resuspended at a concentration of 10⁷ cells/ml in PBS. Cells were then fixed by adding 0.5 ml of cell suspension to 4.5 ml 70% ice-cold ethanol. After 2 h on ice, cells were pelleted and the ethanol was thoroughly decanted. The cell pellet was rinsed in 5 ml PBS, centrifuged, resuspended in 1 ml of PBS containing 0.1% Triton X-100, 0.2 mg/ml DNase-free RNase A, and 20 μg/ml propidium iodide (PI) and 2 μg/ml hoechst-A and incubated at 37° C. for 15 min before FACS analysis. Cells were analyzed using Becton Dickinson Cell Quest FACStation software (version 3.0.1) operating a Becton Dickinson FACSCalibur FACS machine. Gating was used to remove debris and doublets before collection. Results were quantitated using ModFit LT (1999; Topsham, Me.: Verity Software House, Inc.). PI and Hoechst excitation was at 0.488 nm (100 mW) and 351/363 nm (40 mW), respectively. As shown in FIG. 7, treatment with Cpd 1 was observed to induce DNA fragmentation (95% in the presence of Cpd 1 versus only 1.4% in the absence of Cpd 1).

### Example 3

### Protein Expression and Localisation Following Treatment with Cpd 1

After treatment with Cpd 1, protein was extracted from cells and measured as previously described (Kamsteeg et al., 2003). For separation of the cytoplasmic and mitochondrial fractions, cell pellets were processed using the ApoAlert Cell Fractionation kit (Promega) according to the manufacturer's instructions. 20 μg protein was denatured in sample buffer (2.5% sodium dodecyl sulfate [SDS], 10% glycerol, 5% β-mercapto-ethanol, 0.15 M Tris, pH 6.8, and 0.01% bromophenol blue) and subjected to 12% SDS polyacrylamide gel electrophoresis (SDS-PAGE) as previously described (Kamsteeg et al., 2003). The following antibodies and concentrations were used: mouse anti-Bax (BD Biosciences, 1:500), rabbit anti-actin (Sigma, 1:10,000), rabbit anti-cytochrome c (BD Biosciences, 1:1,000), mouse anti-Cox-4 (BD Biosciences, 1:500), rabbit anti-beclin 1 (Santa Cruz Biotechnology, 1:600), mouse anti LC3 (Nanotools, 1:500). These dilutions have been found to be the optimum dilution for each respective antibody. Proteins were observed using enhanced chemiluminescence (Pierce, Rockford, Ill.).

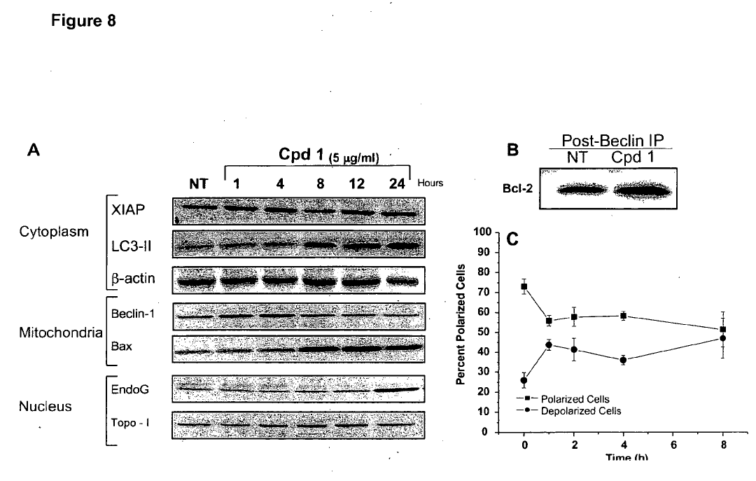

As shown in FIG. 8A, Cpd 1-induced cell death involves upregulation of the autophagic markers, LC3-II and Beclin 1. Western blot analysis on lysate preparations (20 μg protein) of R-182 cells treated with Cpd 1 (5 μg/ml) over 24 hr demonstrated that LC3-II expression, a marker of autophagosome formation and a hallmark of autopaghy, was increased over time. Further, Cpd 1-induced cell death involves Bax and Beclin-1 translocation to the mitochondria (FIG. 8A). R-182 cells were treated with Cpd 1 (5 μg/ml) over 24 hr and the cytoplasmic and mitochondrial fractions separated. Western blot analyses of mitochondrial extracts demonstrate that mitochondrial content of beclin-1 is transiently increased in expression due to its short half life and the expression of the proapoptotic marker Bax is markedly upregulated over time.

Importantly, Beclin 1 immuno-precipitation studies using Cpd 1 treated cell lysates demonstrated that Beclin and Bcl-2 (a member of the BH3 protein family involved in mitochondrial membrane stabilisation) co-precipitate at higher concentrations compared to control showing that upregulated Beclin 1 is able to sequester and inactivate Bcl-2 (FIG. 8B). Further, the over-expression of Bax and Beclin 1 results in mitochondrial membrane depolarization (FIG. 8C). To measure membrane depolarization cells were treated with Cpd 1 as described above, pelleted and washed in HBSS and the cell pellet resuspended in the reaction buffer for JC-1 (Biovision, Inc. MitoCapture™ K250-100). HBSS-washed cell pellets were resuspended in 1 mL aliquots of the Mitocapture reagent buffer with 1 μL of JC-1 solution in DMSO (Biovision). After incubation at 37° C., 5% CO₂ for 20 min, the cells were harvested, washed with Mitocapture reagent buffer and analysed by flow cytometry (λ_{excit}=488 nm and (λₑₘᵢₜ=530 nm for green fluorescence (depolarized) or 590 nm for red fluorescence (polarized). 10,000 gated events were measured with a BD FACScalibur. The inventors conclude that the observed mitochondrial membrane depolarisation (FIG. 8C) permits the translocation of endonuclease G from the mitochondria to the nucleus (FIG. 8A) where it initiates DNA degradation and cell death. Unlike dehydroequol and triphendiol, XIAP expression remains unchanged (FIG. 8A) thereby explaining why apoptosis was not induced.

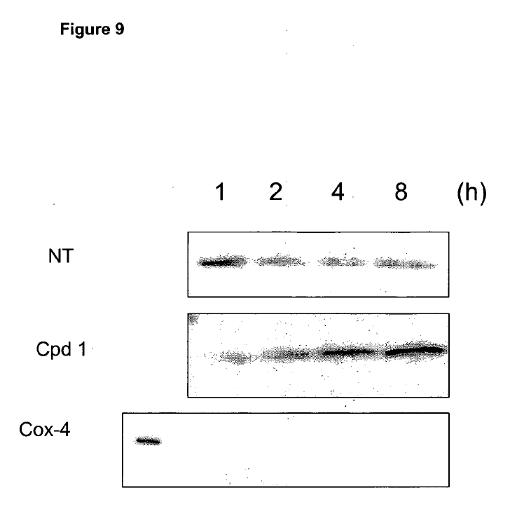

Cpd 1-induced cell death also involves cytochrome c translocation to the cytoplasm (FIG. 9). R-182 cells were treated with Cpd 1 (5 μg/ml) over 8 hr and the cytoplasmic and mitochondrial fractions separated. Western blot analyses of cytoplasmic extracts demonstrate that the occurrence of cytochrome in the cytoplasm is increased over time when compare to no treatment controls.

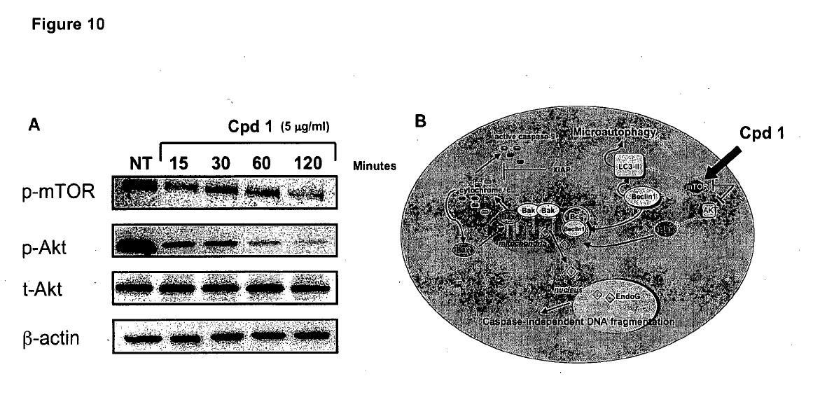

The expression of upstream regulators of autophagy are also modulated in response to Cpd 1. Specifically, as shown in FIG. 10A, mTOR expression and Akt phosphorylation was reduced up to 120 minutes post exposure to Cpd 1. In contrast, total Akt expression remained unchanged.

Without wishing to be bound by any one theory of the mode of action of the autophagic process induced by Cpd 1, the inventors suggest that (as illustrated in FIG. 10B) it would appear to operate primarily via disruption of an as yet unidentified upstream signalling pathway with AKT as substrate. As a consequence of Akt deactivation, mTOR expression is reduced and Bax is upregulated. Reduced mTOR activity enables the activation of Beclin 1 expression which translocates to the mitochondria and sequesters Bcl-2 thereby destabilising the mitochondrial membrane. Reduced mTOR activity also enables LC3-II to participate in the construction of autolysosomes which in combination with the expression of beclin are the typical hallmarks of autophagic cell death. Bax localisation to the mitochondria causes mitochondrial depolarisation resulting in cytochrome c release and endonuclease G translocation to the nucleus thereby initiating DNA degradation and cell death. Cytochrome c release fails to activate intrinsic apoptosis cascade via caspase 9 due to its inhibition of executioner caspases by x-linked inhibitor of apoptosis protein (XIAP).

### Example 4

### In Vivo Demonstration of Cpd 1-Induced Autophagic Cell Death

In vivo studies using tumour-bearing mice were conducted to further confirm that Cpd 1 induces programmed cell death via an autophagic process. SCID mice bearing chemoresistant epithelial ovarian cancer (EOC) xenografts derived from a primary cell culture of a patient's ascites were administered with increasing doses of Cpd 1 by intraperotineal injection resulting in reductions in both tumor volume and tumor weight (data not shown). Representative tumours excised from these mice and tumour-bearing mice administered vehicle control were then fixed, sectioned and subjected to immunohistopathology analysis using an α-enodonuclease G directed monoclonal antibody. Tumor samples were blocked with either 10% horse or goat serum in PBS for 1 hour at room temperature. Following three washes with PBS, samples were incubated overnight at 4° C. with either the anti-EndoG (LifeSpan Bioscience) antibody or mouse IgG1 isotype as negative controls. After three washes with PBS, specific staining was detected by incubating with a peroxidase-conjugated horse anti-mouse antibody (1:1000 dilution) for 1 hour followed by a five-minute incubation with DAB substrate (Vector Laboratories). Tissue sections were then counterstained with haematoxylin (Sigma Chemical Co.) before dehydration with ethanol and Histosolve (Shandon Inc., Pittsburgh, Pa.). Slides were then mounted with Permount (Fisher Scientific, Pittsburgh, Pa.) and visualized by light microscopy.

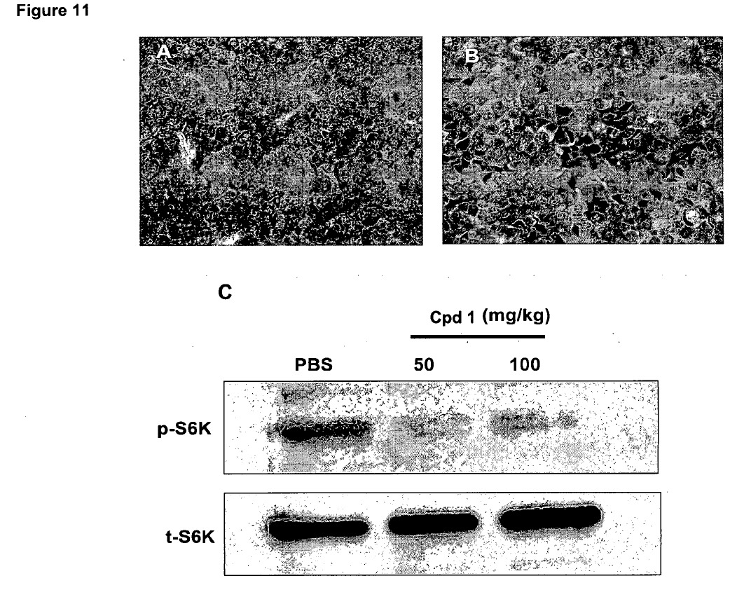

These data demonstrate that the expression of nuclear endonuclease G was increased in tumour sections taken from those animals dosed with Cpd 1 (100 mg/kg) compared to control where only cytoplasmic immunoreactivity was observed (FIGS. 11A and B). These data confirm translocation of endonuclease G from the cytoplasm to nucleus in tumour cells taken from animals dosed with Cpd 1. Further, caspase 3 activity was observed only in tumour sections taken from mice dosed with dehydroequol (not shown) further confirming that Cpd 1 induces caspase-independent death.

A downstream target of mTOR is the ribosomal protein S6K kinase thought to have a role in tumour invasiveness, motility and angiogenesis as well as other degenerative diseases such as diabetes (Dann et al., 2007). Western blot analysis of tumour extracts derived from tumour-bearing mice dosed with Cpd 1 revealed that the phosphorylation status of S6K (S6K-P) was reduced compared to control thereby indicating that mTOR activity is also reduced (FIG. 11C). Tumour tissue was homogenised in lysis buffer containing 1% Nonidet P-40, 0.1% SDS, 0.5% deoxycholate, 150 mM NaCl, 50 mM Tris-HCl pH7.5, 1 mM EDTA, 1 mM PMSF and protease inhibitors (Roche) on ice for 30 minutes. After centrifugation at 15,000×g for 15 minutes, soluble extract was were aliquoted into a separate tube, protein content assayed by the BCA method (Pierce), and 50 μg protein loaded on SDS-PAGE gels. Proteins were electrophoretically transferred to PVDF membranes (Millipore Immobilon P) and the membranes were probed with antibodies specific to p-S6K and t-S6K (LifeSpan Bioscience). The blots were developed on X-ray films using HRP-conjugated secondary antibodies and a chemiluminescent substrate (ECL, Amersham, N.Y.).

These in vivo data correlate with western blot mechanistic studies where it was demonstrated that, when compared to control, endonuclease G expression was increased in nuclear extracts, and phosphorylated mTOR levels were decreased in soluble extracts of cells treated with Cpd 1 in vitro (see FIGS. 8 and 10).

### REFERENCES

Dann S G, Selvaraj A, Thomas G. (2007) mTOR Complex1-S6K1 signaling: at the crossroads of obesity, diabetes and cancer. Trends Mol. Med. 13:252-9
Kamsteeg M, Rutherford T, Sapi E, at al., (2003) Phenoxodiol—an isoflavone analog induces apoptosis in chemoresistant ovarian cancer cells, Oncogene. 22:2611-2620
Kelekar, A., (2005) Autophagy, Ann. N.Y. Acad. Sci. 1066: 259-271
Kondo Y, Kanzawa T, Sawaya R, Kondo S. (2005) The role of autophagy in cancer development and response to therapy. Nat. Rev. Cancer. 5:726-34.
Nixon, R. A., (2006) Autophagy in neurodegenerative disease: friend, foe or turncoat?, Trends Neurosci., 29(9): 528-535
Schrijvers, D. J., et al (2007) Phagocytosis in atherosclerosis: Molecular mechanisms and implications for plaque progression and stability, Cardiovasc. Res. 73(3): 470-80

1. A method for inducing or promoting autophagy in a cell, the method comprising exposing to the cell an effective amount of a compound of formula (I) 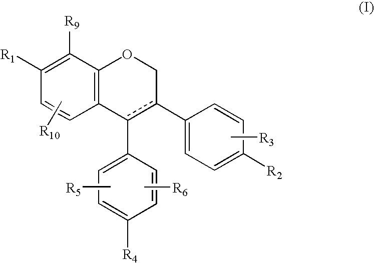
wherein
R₁ is hydrogen, hydroxy, alkyl, alkoxy, halo or OC(O)R₇,
R₂ and R₃ are independently hydrogen, hydroxy, alkoxy, alkyl, cycloalkyl, halo or OC(O)R₇,
R₄, R₅ and R₆ are independently hydrogen, hydroxy, alkoxy, alkyl, cycloalkyl, acyl, amino, C₁₋₄-alkylamino or di(C₁₋₄-alkyl)amino, OC(O)R₇ or OR₈,
R₇ is hydrogen, alkyl, cycloalkyl, aryl, arylalkyl or amino, and
R₈ is aryl or arylalkyl,
R₉ and R₁₀ are independently hydrogen, hydroxy, alkyl, alkoxy or halo, and the drawing [symbol] represents a single bond or a double bond,
or a pharmaceutically acceptable salt or derivative thereof.

2. The method of claim 1 wherein the cell is not a cancer cell.

3. The method of claim 1 wherein the cell is selected from a neuronal cell, myocardial cell, muscle cell or liver cell.

4. The method of any one of claims 1 to 3 wherein the compound is 3-(4-hydroxyphenyl)-4-(4-methoxyphenyl)-8-methylchroman-7-ol.

5. A method for the treatment or prevention of a disease or disorder in a subject, the method comprising administering to the subject an effective amount of a compound of formula (I), or a pharmaceutically acceptable salt or derivative thereof, optionally in association with a carrier and/or excipient, wherein the compound induces or promotes autophagy in at least one cell in the subject.

6. The method of claim 5 wherein the cell is not a cancer cell.

7. The method of claim 5 wherein the cell is selected from a neuronal cell, myocardial cell, muscle cell or liver cell.

8. The method of any one of claims 5 to 7 wherein the compound is 3-(4-hydroxyphenyl)-4-(4-methoxyphenyl)-8-methylchroman-7-ol.

9. The method of any one of claims 5 to 8 wherein the disease or disorder is associated with defective, impaired or otherwise aberrant autophagy or autophagic processes.

10. The method of any one of claims 5 to 9 wherein the cell is a neuronal cell and the method comprises preventing neuronal cell death.

11. The method of any one of claims 5 to 9 wherein the cell is a smooth muscle cell and the method comprises maintaining atherosclerotic plaque stability.

12. An agent for the treatment or prevention of a disease or disorder, the agent comprising a compound of formula (I) or a pharmaceutically acceptable salt or derivative thereof.

13. Use of a compound of formula (I) for the manufacture of a medicament for inducing or promoting autophagy in a cell.

14. Use of a compound of formula (I) for the manufacture of a medicament for treating or preventing a disease or disorder, wherein the compound induces or promotes autophagy in at least one cell of the subject.

15. A composition when used for inducing or promoting autophagy in a cell, the composition comprising a compound of formula (I) or a pharmaceutically acceptable salt or derivative thereof, optionally in association with a carrier and/or excipient.

16. A composition when used for treating or preventing a disease or disorder in a subject, the composition comprising a compound of formula (I), or a pharmaceutically acceptable salt or derivative thereof, optionally in association with a carrier and/or excipient, wherein the compound induces or promotes autophagy in at least one cell in the subject.
